SEQUENCE LISTING (47 sequences)

SEQ ID NO: 1 - protein, 11 aa, synthetic construct
EVQGLFPFPRV

SEQ ID NO: 2 - protein, 9 aa, synthetic construct
QGLIPFPRV

SEQ ID NO: 3 - protein, 9 aa, synthetic construct
QGLIPFPRV

SEQ ID NO: 4 - protein, 8 aa, synthetic construct
GLFPFPRV

SEQ ID NO: 5 - protein, 8 aa, synthetic construct
GLIPFPRV

SEQ ID NO: 6 - protein, 7 aa, synthetic construct
LFPFPRV

SEQ ID NO: 7 - protein, 7 aa, synthetic construct
LIPFPRV

SEQ ID NO: 8 - protein, 6 aa, synthetic construct
IPFPRV

SEQ ID NO: 9 - protein, 6 aa, synthetic construct
FPFPRV

SEQ ID NO: 10 - protein, 20 aa, synthetic construct
DLVTQVLQPGQTGVWFGPRL

SEQ ID NO: 11 - protein, 12 aa, synthetic construct
VASWMPSSSPRL

SEQ ID NO: 12 - protein, 9 aa, synthetic construct
DSASFTPRL

SEQ ID NO: 13 - protein, 12 aa, synthetic construct
SEGNLVNFTPRL

SEQ ID NO: 14 - protein, 12 aa, synthetic construct
GANMGLYAFPRV

SEQ ID NO: 15 - protein, 10 aa, synthetic construct
ASGLVAFPRV

SEQ ID NO: 16 - protein, 10 aa, synthetic construct
DAGLFPFPRV

SEQ ID NO: 17 - protein, 9 aa, synthetic construct
EQLIPFPRV

SEQ ID NO: 18 - protein, 12 aa, synthetic construct
DGVLNLYPFPRV

SEQ ID NO: 19 - protein, 8 aa, synthetic construct
QLYAFPRV

SEQ ID NO: 20 - protein, 12 aa, synthetic construct
DGVLSLYPFPRV

SEQ ID NO: 21 - protein, 16 aa, synthetic construct
GSESTDSTSMWFGPRL

SEQ ID NO: 22 - protein, 10 aa, synthetic construct
SAGLVAYPRI

SEQ ID NO: 23 - protein, 8 aa, synthetic construct
KSDLFPRL

SEQ ID NO: 24 - protein, 10 aa, synthetic construct
TFGIIQKPRV

SEQ ID NO: 25 - protein, 14 aa, synthetic construct
VFYTKSDDNDYPRI

SEQ ID NO: 26 - protein, 14 aa, synthetic construct
GIFTQSAHGSYPRV

SEQ ID NO: 27 - protein, 8 aa, synthetic construct
GLYAFPRV

SEQ ID NO: 28 - protein, 7 aa, synthetic construct
LYAFPRV

SEQ ID NO: 29 - protein, 6 aa, synthetic construct
YAFPRV

SEQ ID NO: 30 - protein, 9 aa, synthetic construct
SGLVAFPRV

SEQ ID NO: 31 - protein, 8 aa, synthetic construct
GLVAFPRV

SEQ ID NO: 32 - protein, 7 aa, synthetic construct
LVAFPRV

SEQ ID NO: 33 - protein, 6 aa, synthetic construct
VAFPRV

SEQ ID NO: 34 - protein, 15 aa, synthetic construct
NGASGNGGLWFGPRL

SEQ ID NO: 35 - protein, 14 aa, synthetic construct
NDFFLKAAKSVPRI

SEQ ID NO: 36 - protein, 6 aa, synthetic construct
YFSPRL

SEQ ID NO: 37 - protein, 11 aa, synthetic construct
DTSGLIPFPRV

SEQ ID NO: 38 - protein, 5 aa, synthetic construct
PFPRV

SEQ ID NO: 39 - protein, 4 aa, synthetic construct
AFPR

SEQ ID NO: 40 - protein, 4 aa, synthetic construct
FPRV

SEQ ID NO: 41 - protein, 4 aa, synthetic construct
FPRV

SEQ ID NO: 42 - protein, 8 aa, synthetic construct
CFPFPRVC

SEQ ID NO: 43 - protein, 9 aa, synthetic construct
CLFPFPRVC

SEQ ID NO: 44 - protein, 10 aa, synthetic construct
CGLFPFPRVC

SEQ ID NO: 45 - protein, 8 aa, synthetic construct
CIPFPRVC

SEQ ID NO: 46 - protein, 9 aa, synthetic construct
CLIPFPRVC

SEQ ID NO: 47 - protein, 10 aa, synthetic construct
CGLIPFPRVC

(19) United States
(12) Patent Application Publication
(10) Pub. No.: US 2026/0047574 A1
(43) Pub. Date: Feb. 19, 2026

(54) BIOACTIVE PEPTIDES FOR CONTROLLING INSECTS

(71) Applicant: The United States of America, as represented by the Secretary of Agriculture, Washington, DC (US)

(72) Inventors: MAN-YEON CHOI, ALBANY, OR (US); RYSSA PARKS, CORVALLIS, OR (US); BRIANA E. PRICE, CORVALLIS, OR (US); DANIEL K. HASEGAWA, SALINAS, CA (US)

(21) Appl. No.: 18/802,427

(22) Filed: Aug. 13, 2024

(51) Int. Cl. A01N 37/46; A01N 25/00; A01N 25/06; C07K 7/06; C07K 7/08

(52) U.S. Cl. CPC A01N 37/46; A01N 25/006; A01N 25/06; C07K 7/06; C07K 7/08

(57) ABSTRACT

Disclosed are synthetic peptides developed from the western flower thrip (Frankliniella occidentalis), spotted-wing drosophila (Drosophila suzukii), diamondback moth (Plutella xylostella), lygus (Lygus Hesperus), and other insect neuropeptides. Peptides disclosed can be used to repel, control, or deter Frankliniella spp., Drosophila spp., Plutella ssp., and Lygus ssp., including Frankliniella occidentalis, Drosophila suzukii, Plutellaxylostella, and Lygus Hesperus from feeding on agricultural and horticultural plants. The peptides can be combined with bait materials, or applied directly to plants or areas.

### INCORPORATION BY REFERENCE STATEMENT

An XML file for a “Sequence Listing XML”, submitted electronically using the USPTO Patent Center and having the file name: “Sequence_Listing-0072.23.xml”, creation date: Nov. 18, 2024, and file size: 41,474 bytes, is herein incorporated by reference.

### BACKGROUND OF THE INVENTION

### Field of Invention

Disclosed are synthetic peptides developed from the western flower thrip (Frankliniella occidentalis), spotted-wing drosophila (Drosophila suzukii), diamondback moth (Plutella xylostella), lygus (Lygus Hesperus), and other insect neuropeptides. Peptides disclosed can be used to repel, control, or deter Frankliniella spp., Drosophila spp., Plutella ssp., and Lygus ssp., including Frankliniella occidentalis, Drosophila suzukii, Plutella xylostella, and Lygus Hesperus from feeding on agricultural and horticultural plants. The peptides can be combined with bait materials, or applied directly to plants or areas.

### Background

The infestations and resulting damage/problems associated with thrips and fly pests are increasing every year. Thrips have hundreds of host plants, including many ornamental and nursery crops. One of the most economically important pests is the western flower thrips (WFT), Frankliniella occidentalis, owing to its serious damage on greenhouse, horticultural and nursery crops. Not only direct damage from feeding on flowers and fruits, WFT also transmit tomato spotted wilt virus (TSWV) that is economically the most important. The combined value in 2022 of two of the most important vulnerable commodities i.e., greenhouse and nursery according to USDA and Oregon Department of Agriculture (ODA) is $1.4 billion in the Oregon alone. Those economic impacts are increasing every year. Today, WFT can be found on nearly all continents, making them one of the most economically significant pests, globally. Current control for thrips primarily relies on chemical insecticides despite causing potential negative effects to human health and environmental degradation as well as development of insecticide resistance.

Spotted-wing drosophila (SWD), Drosophila suzukii, is a severe invasive pest attacking a wide range of ripening fruits including grapes, cherries and all berry crops. SWD management has recently been ranked a top priority among small fruit growers. The infestation areas of SWD have been rapidly expanding across the U.S., Canada, and Europe. The estimated economic impact is $800 million per year in the U.S. alone, and increasing every year. Currently, the only way to effectively control this destructive pest is through the use of chemical insecticides despite negative effects on environmental and human health, and potential development of chemical resistance.

Therefore, there is a strong need to develop environmentally friendly alternatives for WFT and SWD control. As presented herein, we have developed novel bioactive peptides that have broad applications, well beyond the thrips and fly pests. For example, the novel bioactive peptides in accordance with some embodiments of the present invention are commercially viable and specific for western flower thrips, spotted-wing drosophila, diamondback moth, and lygus.

Insect neuropeptides (NPs) are part of a large group of neurohormones that regulate important biological functions and are found in invertebrates. A variety of peptide families from insects have been identified and classified by their core structures and functionalities. These neuropeptide ligands bind to G-Protein-coupled receptors (GPCRs), a large group of signaling receptors for various signal transductions. GPCRs are membrane embedded proteins, also known as 7 transmembrane receptors, activated by a wide variety of stimulants including light, odorant molecules, peptide and non-peptide neurotransmitters, hormones, growth factors and lipids. They control a wide variety of physiological processes including sensory transduction, cell-cell communication, neuronal transmission, and hormonal signaling.

The PRXamide (X=any amino acid) family of neuropeptides is based on the core amino acid sequence at the C-terminal end that are required for activity and on sequence homology of their GPCRs. The PRXamide (PRX-NH2) family of neuropeptides are ubiquitously found in invertebrate animals. The family includes the pyrokinin, pheromone biosynthesis activating neuropeptide (PBAN), diapause hormone (DH), capability (CAPA), and ecdysis triggering hormone (ETH) throughout Arthropod and other invertebrate. Knowledge about the structure of specific peptide ligands and their receptors is necessary to more fully understand their interactions to facilitate the development of antagonists and agonists that can be developed for controlling potential biological targets.

We identified a variety of NPs from the western flower thrips (Frankliniella occidentalis), spotted-wing drosophila (Drosophila suzukii), diamondback moth (Plutella xylostella), lygus (Lygus Hesperus), and other insect neuropeptides, characterized by the natural ligands and short synthetic peptides. In addition, we identified four insect GPCRs, determined neuropeptide binding affinities using an insect Sf9 cell expression system, and discovered short bioactive peptides strongly binding to their GPCRs. These bioactive peptides offer great potential to develop biologically-based control methods for agricultural important insect pests.

### SUMMARY OF THE INVENTION

Disclosed are synthetic peptides developed from the western flower thrip (Frankliniella occidentalis), spotted-wing drosophila (Drosophila suzukii), diamondback moth (Plutella xylostella), lygus (Lygus Hesperus), and other insect neuropeptides. Peptides disclosed can be used to repel, control, or deter Frankliniella spp., Drosophila spp., Plutella ssp., and Lygus ssp., including Frankliniella occidentalis, Drosophila suzukii, Plutella xylostella, and Lygus Hesperus from feeding on agricultural and horticultural plants. The peptides can be combined with bait materials, or applied directly to plants or areas.

The novel bioactive peptides in accordance with some embodiments of the present invention will have potential benefits/advantages:
1. Bioactive peptides will not affect human health and environment.
2. The bioactive peptides can be mixed with conventional or biological insecticides to increase insecticidal efficacy.
3. Bioactive peptides by themselves or in combination with conventional insecticides will reduce the current chemical dose sprayed in the field.

Disclosed herein is an insecticide composition for controlling an insect, the composition comprising an effective amount of peptide having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, SEQ ID NO: 42, SEQ ID NO: 45, or a combination thereof. In one embodiment of the invention, the insecticide composition for controlling insects, comprises an effective amount of peptide having SEQ ID NO: 4, SEQ ID NO: 5, SEQ ID NO: 6, SEQ ID NO: 7, SEQ ID NO: 8, SEQ ID NO: 9, SEQ ID NO: 28, or a combination thereof. In another embodiment of the invention, the insecticide composition further compromises an insecticide carrier. In one particular embodiment, the carrier is saline. In another embodiment of the invention, the insecticide composition further compromises one or more suitable propellants, carriers, diluents, adjuvants, preservatives, dispersants, solvents, or emulsifying agents.

Disclosed herein is a spray composition for controlling an insect, the spray comprises of: (a) an effective amount of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, SEQ ID NO: 42, SEQ ID NO: 45, or a combination thereof and (b) a propellant.

Disclosed herein is a bait composition for controlling an insect, the bait comprises of: (a) an effective amount of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, SEQ ID NO: 42, SEQ ID NO: 45, or a combination thereof, (b) one or more food materials; and (c) optionally a phagostimulant.

Disclosed herein is an insecticide composition for controlling Frankliniella ssp., the composition comprises of an effective amount of peptide having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 42, SEQ ID NO: 45, or a combination thereof. In one embodiment of the invention, the insecticide composition is for controlling Frankliniella occidentalis.

Disclosed herein is an insecticide composition for controlling Drosophila ssp., the composition comprises of an effective amount of peptide having SEQ ID NO: 2, SEQ ID NO: 4, SEQ ID NO: 5, SEQ ID NO: 6, SEQ ID NO: 7, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 28, SEQ ID NO: 32, or a combination thereof. In one embodiment of the invention, the insecticide composition is for controlling Drosophila suzukii.

Disclosed herein is an insecticide composition for controlling Plutella ssp., the composition comprises of an effective amount of peptide having SEQ ID NO: 1, SEQ ID NO: 2, SEQ ID NO: 4, SEQ ID NO: 5, SEQ ID NO: 6, SEQ ID NO: 7, SEQ ID NO: 14, SEQ ID NO: 15, SEQ ID NO: 16, SEQ ID NO: 18, SEQ ID NO: 19, SEQ ID NO: 20, SEQ ID NO: 27, SEQ ID NO: 28, SEQ ID NO: 30, SEQ ID NO: 31, SEQ ID NO: 32, or a combination thereof. In one embodiment of the invention, the insecticide composition is for controlling Plutella xylostella.

Disclosed herein is an insecticide composition for controlling Lygus ssp., the composition comprises of an effective amount of peptide having SEQ ID NO: 4, SEQ ID NO: 5, SEQ ID NO: 6, SEQ ID NO: 7, SEQ ID NO: 8, SEQ ID NO: 9, SEQ ID NO: 17, SEQ ID NO: 19, SEQ ID NO: 22, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, or a combination thereof. In one embodiment of the invention, the insecticide composition is for controlling Lygus hesperus.

Also disclosed is a method for controlling an insect spp., the method comprises of contacting an insect or its environment with a biologically effective amount of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, SEQ ID NO: 42, SEQ ID NO: 45, or a combination thereof, wherein the mortality of said insect increases. In one embodiment of the invention, the insect is Frankliniella occidentalis. In another embodiment of the invention, the insect is Drosophila suzukii. In yet another embodiment of the invention, the insect is Plutella xylostella. In still another embodiment of the invention, the insect is Lygus hesperus.

An additional embodiment of the invention disclosed herein is an insecticide composition for controlling an insect, the composition comprising an effective amount of peptide having SEQ ID NO: 38, SEQ ID NO: 39, SEQ ID NO: 40, SEQ ID NO: 41, SEQ ID NO: 43, SEQ ID NO: 44, SEQ ID NO: 46, SEQ ID NO: 47, or a combination thereof.

This summary is provided to introduce a selection of concepts in a simplified form that are further described below in the detailed description. This summary is not intended to identify key features of the claimed subject matter, nor is intended as an aid in determining the scope of the claimed invention.

### BRIEF DESCRIPTION OF THE DRAWINGS

Embodiments will hereinafter be described in conjunction with the appended drawings, where like designations denote like elements.

FIGS. 1A and 1B depict dosage-response of CAPA and short peptides on the CAPA GPCR identified from western flower thrips, Frankliniella occidentalis, according to one or more embodiments. FIG. 1A shows dosage-response of CAPA-PVK1 (EVQGLFPFPRV) (SEQ ID NO:1) and its modified bioactive peptides (CAPA-PVK1-1 (FPFPRV) (SEQ ID NO: 9), CAPA-PVK1-2 (LFPFPRV) (SEQ ID NO: 6), and CAPA-PVK1-3 (GLFPFPRV) (SEQ ID NO: 4)). FIG. 1B shows dosage-response of CAPA-PVK2 (QGLIPFPRV) (SEQ ID NO: 2) and its modified bioactive peptides (CAPA-PVK2-1 (IPFPRV) (SEQ ID NO: 8), CAPA-PVK2-2 (LIPFPRV) (SEQ ID NO: 7), and CAPA-PVK2-3 (GLIPFPRV) (SEQ ID NO: 5)).

FIG. 2 depicts mortality of female thrips within 24 h after injection of bioactive peptides of western flower thrips, Frankliniella occidentalis, according to one or more embodiments. CAPA-PVK2 (QGLIPFPRV) (SEQ ID NO: 2) (labeled in FIG. 2 as FraocCAPA2) (1 nmol) or CAPA-PK2-1 (VASWMPSSSPRL) (SEQ ID NO: 11) (labeled in FIG. 2 as FraocPK2-1) (0.5 nmol) were dissolved in 5 nL water. Control: 5 nL water. Three replicates per each treatment.

FIG. 3 depicts feeding assays with and without pollen as a phagostimulant in thrips, according to one or more embodiments.

FIGS. 4A and 4B depict survival (%) of Frankliniella occidentallis over 72 hours when fed various peptide solutions from two different delivery methods, according to one or more embodiments. FIG. 4A shows survival (%) of Frankliniella occidentallis over 72 hours when fed various peptide solutions without pollen as a phagostimulant. FIG. 4B shows survival (%) of Frankliniella occidentallis over 72 hours when fed various peptide solutions with pollen as a phagostimulant. As shown in FIG. 4B, thrips fed CAPA-PVK1-1 (FPFPRV) (SEQ ID NO: 9) peptide mixed in the cotyledon and pollen showed mortality at 60% within 48 h and 70% within 72 h, respectively.

FIGS. 5A and 5B depict a feeding assay with bioactive peptides on thrips (FIG. 5A) and percentage of thrips survival (FIG. 5B), according to one or more embodiments. Thrips fed on 1% sucrose solution containing 10 nmol of bioactive peptide and mortality was checked daily for two weeks. The bioactive peptides contained in the droplet of 1% sucrose solution included CAPA-PVK1-1 (FPFPRV) (SEQ ID NO: 9), CAPA1-1C (CFPFPRVC) (SEQ ID NO: 42), CAPA-PVK2-1 (IPFPRV) (SEQ ID NO: 8), CAPA2-1C (CIPFPRVC) (SEQ ID NO: 45), or an unrelated peptide from Meloidogyne incognita (PGVLRF). The droplet of solution typically lasts about 5 days. The droplet of solution normally lasted 5 days and was replaced with a new droplet of solution, but was checked daily to decide whether to replace the droplet before or after 5 days. Water only without sucrose was used as negative control. Thrips were starved 1 h prior to feeding. Five replicates. Survival rates were compared between treatments using log-rank analysis in SAS 9.4.

FIG. 6 depicts dosage-response of CAPA peptides on the CAPA GPCR identified from spotted-wing drosophila (SWD), Drosophila suzukii, according to one or more embodiments. As shown in FIG. 6, SWD peptides (CAPA-PVK1 (GANMGLYAFPRV) (SEQ ID NO: 14) (labeled in FIG. 6 as DrosuCAPA1) and CAPA-PVK2 (ASGLVAFPRV) (SEQ ID NO: 15) (labeled in FIG. 6 as DrosuCAPA1)) from Drosophila suzukii, as well as thrip peptide (CAPA-PVK2 (QGLIPFPRV) (SEQ ID NO: 2) (labeled in FIG. 6 as FraocCAPA2)) from Frankliniella occidentalis will be agonists of the natural peptide ligands for SWD receptors and interfere with the normal functioning of the peptides and their receptors.

FIG. 7 depicts mortality of spotted-wing drosophila (Drosophila suzukii) adults within 24 h after the feeding of the non-nutritive sugars and injecting the bioactive peptide, according to one or more embodiments. The flies were injected with a 100 pmol of the bioactive peptide CAPA-PVK2 (ASGLVAFPRV) (SEQ ID NO: 15) (labeled in FIG. 7 as DrosuCAPA2) after 1 h feeding of the diets and fed continuously for 24 h. S: 0.5M sucrose; E+S: 0.5M erythritol+0.5M sucrose; S+Sul: 0.5M sucrose+0.5M sucralose; E+Sul: 0.5M erythritol+0.5M sucralose.

### BRIEF DESCRIPTION OF THE SEQUENCES

SEQ ID NO 1: is EVQGLFPFPRV, a synthetically generated peptide.

SEQ ID NO 2: is QGLIPFPRV, a synthetically generated peptide.

SEQ ID NO 3: is QGLIPFPRV**, a synthetically generated peptide, wherein ** denotes that the C-terminal end of the peptide is acidified (COOH).

SEQ ID NO 4: is GLFPFPRV, a synthetically generated peptide.

SEQ ID NO 5: is GLIPFPRV, a synthetically generated peptide.

SEQ ID NO 6: is LFPFPRV, a synthetically generated peptide.

SEQ ID NO 7: is LIPFPRV, a synthetically generated peptide.

SEQ ID NO 8: is IPFPRV, a synthetically generated peptide.

SEQ ID NO 9: is FPFPRV, a synthetically generated peptide.

SEQ ID NO 10: is DLVTQVLQPGQTGVWFGPRL, a synthetically generated peptide.

SEQ ID NO 11: is VASWMPSSSPRL, a synthetically generated peptide.

SEQ ID NO 12: is DSASFTPRL, a synthetically generated peptide.

SEQ ID NO 13: is SEGNLVNFTPRL, a synthetically generated peptide.

SEQ ID NO 14: is GANMGLYAFPRV, a synthetically generated peptide.

SEQ ID NO 15: is ASGLVAFPRV, a synthetically generated peptide.

SEQ ID NO 16: is DAGLFPFPRV, a synthetically generated peptide.

SEQ ID NO 17: is EQLIPFPRV, a synthetically generated peptide.

SEQ ID NO 18: is DGVLNLYPFPRV, a synthetically generated peptide.

SEQ ID NO 19: is QLYAFPRV, a synthetically generated peptide.

SEQ ID NO 20: is DGVLSLYPFPRV, a synthetically generated peptide.

SEQ ID NO 21: is GSESTDSTSMWFGPRL, a synthetically generated peptide.

SEQ ID NO 22: is SAGLVAYPRI, a synthetically generated peptide.

SEQ ID NO 23: is KSDLFPRL, a synthetically generated peptide.

SEQ ID NO 24: is TFGIIQKPRV, a synthetically generated peptide.

SEQ ID NO 25: is VFYTKSDDNDYPRI, a synthetically generated peptide.

SEQ ID NO 26: is GIFTQSAHGSYPRV, a synthetically generated peptide.

SEQ ID NO 27: is GLYAFPRV, a synthetically generated peptide.

SEQ ID NO 28: is LYAFPRV, a synthetically generated peptide.

SEQ ID NO 29: is YAFPRV, a synthetically generated peptide.

SEQ ID NO 30: is SGLVAFPRV, a synthetically generated peptide.

SEQ ID NO 31: is GLVAFPRV, a synthetically generated peptide.

SEQ ID NO 32: is LVAFPRV, a synthetically generated peptide.

SEQ ID NO 33: is VAFPRV, a synthetically generated peptide.

SEQ ID NO 34: is NGASGNGGLWFGPRLa, a synthetically generated peptide.

SEQ ID NO 35: is NDFFLKAAKSVPRI, a synthetically generated peptide.

SEQ ID NO 36: is LSD . . . YFSPRLa, a synthetically generated peptide.

SEQ ID NO 37: is DTSGLIPFPRV, a synthetically generated peptide.

SEQ ID NO 38: is PFPRV, a synthetically generated peptide.

SEQ ID NO 39: is AFPR, a synthetically generated peptide.

SEQ ID NO 40: is FPRV, a synthetically generated peptide.

SEQ ID NO 41: is PRV, a synthetically generated peptide.

SEQ ID NO 42: is CFPFPRVC, a synthetically generated peptide.

SEQ ID NO 43: is CLFPFPRVC, a synthetically generated peptide.

SEQ ID NO 44: is CGLFPFPRVC, a synthetically generated peptide.

SEQ ID NO 45: is CIPFPRVC, a synthetically generated peptide.

SEQ ID NO 46: is CLIPFPRVC, a synthetically generated peptide.

SEQ ID NO 47: is CGLIPFPRVC, a synthetically generated peptide.

### DETAILED DESCRIPTION

Neuropeptides are part of a large group of neurohormones that regulate important biological functions and are found in invertebrates. A variety of peptide families from western flower thrip (Frankliniella occidentalis), spotted-wing drosophila (Drosophila suzukii), diamondback moth (Plutella xylostella), lygus (Lygus Hesperus), and other insect neuropeptides have been identified and classified by their core structures and functionalities. These neuropeptide ligands bind to G Protein-coupled receptors (GPCRs), a large group of signaling receptors for various signal transductions. GPCRs are membrane embedded proteins, also known as 7 transmembrane receptors, activated by a wide variety of stimulants including light, odorant molecules, peptide and non-peptide neurotransmitters, hormones, growth factors and lipids. They control a wide variety of physiological processes including sensory transduction, cell-cell communication, neuronal transmission, and hormonal signaling.

The PRXamide (X=any amino acid) family of neuropeptides is based on the core amino acid sequence at the C-terminal end that are required for activity and on sequence homology of their GPCRs (Jurenka, R., Adv. Insect Physiol., (2015) 49:123-70). The PRXamide family of neuropeptides are ubiquitous in invertebrate animals. The family includes proteins such as pyrokinin, pheromone biosynthesis-activating neuropeptide, diapause hormone, CAPA/periviscerokinin (a.k.a. cardioacceleratory peptide 2b), and ecdysis triggering hormone in many arthropods and gastropods. However, knowledge about structure of specific peptide ligands and their receptors is necessary to more fully understand their interactions to facilitate the development of antagonists and agonists.

Disclosed herein are synthetic peptides developed from the western flower thrip (Frankliniella occidentalis), spotted-wing drosophila (Drosophila suzukii), diamondback moth (Plutella xylostella), lygus (Lygus Hesperus), and other insect neuropeptides. Peptides disclosed can be used to repel, control, or deter Frankliniella spp., Drosophila spp., Plutella ssp., and Lygus ssp., including Frankliniella occidentalis, Drosophila suzukii, Plutella xylostella, and Lygus Hesperus from feeding on agricultural and horticultural plants. The peptides can be combined with bait materials, or applied directly to plants or areas.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2^{nd} ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount. For example, about 1.0 g means 0.9 g to 1.1 g and all values within that range, whether specifically stated or not.

“Optional” or “optionally” means that the subsequently described event or circumstance may or may not occur, and that the description includes instances in which said event or circumstance occurs and instances where it does not.

The term “a nucleic acid consisting essentially of”, and grammatical variations thereof, means nucleic acids that differ from a reference nucleic acid sequence by 20 or fewer nucleic acid residues and also perform the function of the reference nucleic acid sequence. Such variants include sequences which are shorter or longer than the reference nucleic acid sequence, have different residues at particular positions, or a combination thereof.

“Activity” of a synthetic peptide, as used herein, refers to the capacity to obtain mortality or paralysis in target insects when such target insects are exposed to the peptides (e.g., via feeding or injection), which mortality or paralysis is significantly higher than a negative control (e.g., a buffer).

“Carrier” as used herein refers to any method of dispersal, dispensation, application, timed-release, encapsulation, microencapsulation, or the like to apply the insect-affecting composition as further described herein. In embodiments, such “carriers” may include a variety of microencapsulation, controlled-release, and other dispersion technologies available to those of ordinary skill in the art.

“Control” or “controlling” as used herein refers to any means for preventing infestation, reducing the population of already infested areas, or elimination of pest population(s) whose “control” is desired. Indeed, “controlling” as used herein refers to any indicia of success in prevention, elimination, reduction, repulsion, or amelioration of a pest population or pest problem.

An “effective amount” is an amount sufficient to effect desired beneficial or deleterious results. In terms of treatment, an “effective amount” is that amount sufficient to make the target pest non-functional by causing an adverse effect on that pest, including (but not limited to) physiological damage to the pest; inhibition or modulation of pest growth; inhibition or modulation of pest reproduction; or death of the pest. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The terms “polypeptide, peptide or protein” refer to polymers in which the monomers are amino acid residues which are joined together through amide bonds. When the amino acids are alpha-amino acids, either the L-optical isomer or the D-optical isomer can be used. The terms are used interchangeably herein. These terms apply to amino acid polymers in which one or more amino acid residues are an artificial chemical mimetic of a corresponding naturally occurring amino acid, as well as to naturally occurring amino acid polymers and non-naturally occurring amino acid polymers.

A “conservative substitution” in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4 or 5 substitutions) retains the structure and function of the wild-type protein or peptide. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartate; or (d) a residue having a bulky side chain, for example, phenylalanine, is substituted for (or by) one not having a side chain, for example, glycine.

The term “phagostimulant” refers to any substance that will entice the target pest to ingest the selected bioactive peptide. Suitable phagostimulants include but are not limited to syrups, honey, aqueous solutions of sucrose, artificial sweeteners such as sucralose, saccharin, and other artificial sweeteners, starch, amino acids, and other proteins. Additionally, the bait material containing the bioactive peptide disclosed herein would be incorporated in water soluble baits, oil-in water or oil/water emulsion baits, liquid type or gel type of baits.

The ready-to-use preparations of phagostimulants can be in the form of a wettable powder, flowable concentrate solution, water soluble granules, ultra-low volume formulation, and the like, which can be applied to the target habitat. Phagostimulants can be used in combination with peptides of the present disclosure to enhance or encourage uptake by target pests. In essence, the combination is an insect bait. Such baits can also include any other component desired by one of skill in the art, such as carriers, preservatives, odorants, molluscicides, insecticides and the like. Phagostimulants can include carbohydrates such as glucose, fructose, arabinose, sorbitol, maltose, glucose, lactose, or any other small sugar. It will be obvious to a person skilled in the art that some carbohydrates and/or amino acids are likely to act as a deterrent. Thus, a bait, or other composition of the present invention can include phagostimulant(s) that attract a target pest and components that repel other animals (such as beneficial insects, pets and wildlife). Such variations are easily appreciated by any person skilled in the art.

The “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman-Wunsch algorithm (Needleman and Wunsch, J Mol Biol, (1970) 48:3, 443-53). A computer-assisted sequence alignment can be conveniently performed using a standard software program such as GAP which is part of the Wisconsin Package Version 10.1 (Genetics Computer Group, Madison, Wis., USA) using the default scoring matrix with a gap creation penalty of 50 and a gap extension penalty of 3.

### Peptides

The peptides provided herein can be synthesized by any suitable method, such as exclusively solid-phase techniques, partial solid-phase techniques, fragment condensation, or classical solution addition. The amino acids of the compounds of the invention are typically joined to adjacent groups through amide linkages. For example, without being limited thereto, the peptide variants may be synthesized by methods well known to those skilled in the art of peptide synthesis, e.g., solution phase synthesis [see Finn and Hoffman, In “Proteins,” Vol. 2, 3rd Ed., H. Neurath and R. L. Hill (eds.), Academic Press, New York, pp. 105-253 (1976)], or solid phase synthesis [see Barany and Merrifield, In “The Peptides,” Vol. 2, E. Gross and J. Meienhofer (eds.), Academic Press, New York, pp. 3-284 (1979)], or stepwise solid phase synthesis as reported by Merrifield [J. Am. Chem. Soc. 85:2149-2154 (1963)], the contents of each of which are incorporated herein by reference. However, the peptide fragments are preferably produced by recombinant DNA techniques, which are particularly suitable for large-scale use.

Synthesis by the use of recombinant DNA techniques, for the purpose of this application, should be understood to include the suitable employment of structural genes coding for the sequence as specified hereinafter. The synthetic peptides may also be obtained by transforming a microorganism or plant using an expression vector including a promoter or operator, or both, together with such structural genes and causing such transformed microorganisms or plant to express the peptide.

Vectors used in practicing the present invention are selected to be operable as cloning vectors or expression vectors in the selected host cell. Numerous vectors are known to practitioners skilled in the art, and selection of an appropriate vector and host cell is a matter of choice. The vectors may, for example, be bacteriophage, plasmids, viruses, or hybrids thereof, such as those described in Sambrook et al. [Molecular Cloning: A Laboratory Manual, Cold Springs Harbor Laboratory, 1989] or Ausubel et al. [Current Protocols in Molecular Biology, John Wiley & Sons, Inc, 1995], the contents of each of which are herein incorporated by reference. Further, the vectors may be non-fusion vectors (i.e., those producing the peptides of the invention not fused to any heterologous polypeptide), or alternatively, fusion vectors (i.e., those producing the peptides fused to a vector encoded polypeptide). The fusion proteins would of course vary with the particular vector chosen.

In certain embodiments, the insects repelled by the compositions or methods disclosed herein are Frankliniella spp., Drosophila spp., Plutella ssp., and Lygus ssp., including Frankliniella occidentalis, Drosophila suzukii, Plutella xylostella, and Lygus Hesperus.

Disruption and/or interference of the normal PRXamide peptide function would result in a variety of negative effects on insect survival and/or development. Some novel aspects and benefits/advantages of the compositions or methods disclosed herein are listed below.

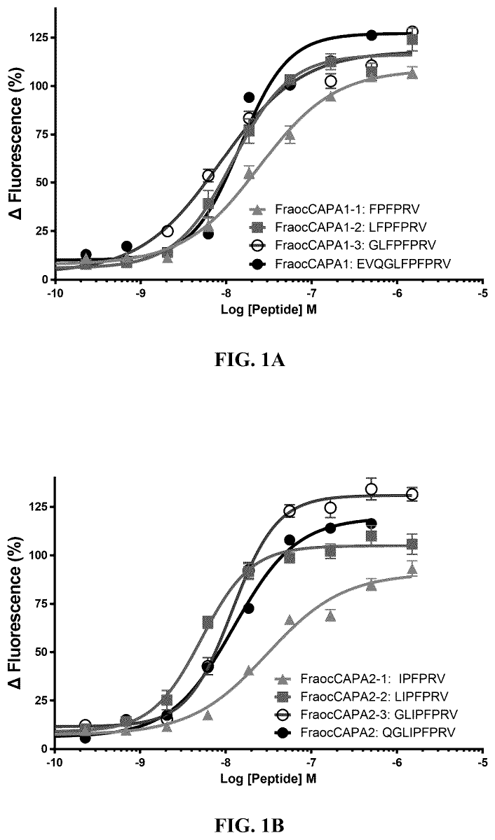

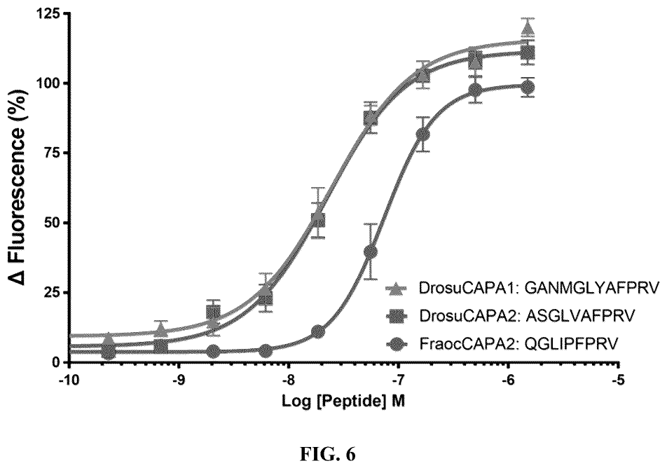

1. Seven short peptides, CAPA-PVK1-3 (GLFPFPRV) (SEQ ID NO: 4), CAPA-PVK2-3 (GLIPFPRV) (SEQ ID NO: 5), CAPA-PVK1-2 (LFPFPRV) (SEQ ID NO: 6), CAPA-PVK2-2 (LIPFPRV) (SEQ ID NO: 7), CAPA-PVK2-1 (IPFPRV) (SEQ ID NO: 8), and CAPA-PVK1-1 (FPFPRV) (SEQ ID NO: 9) each from Frankliniella occidentalis, as well as CAPA-PVK1-2 (LYAFPRV) (SEQ ID NO: 28) from Drosophila suzukii have strong GPCR binding properties (Tables 1-4, below). The peptides will be agonists of the natural peptide ligands for both thrips and SWD receptors and interfere with the normal functioning of the peptides and their receptors (FIGS. 1A, 1B, and 6).

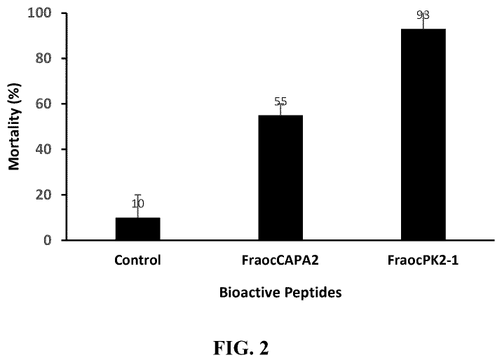

2. Bioactive peptides (CAPA-PVK2 (QGLIPFPRV) (SEQ ID NO: 2) and CAPA-PK2-1 (VASWMPSSSPRL) (SEQ ID NO: 11)) each from Frankliniella occidentalis), when injected into the thrips, have strong detrimental effects, including mortality, spiraling abdomen, and excretion (FIG. 2).

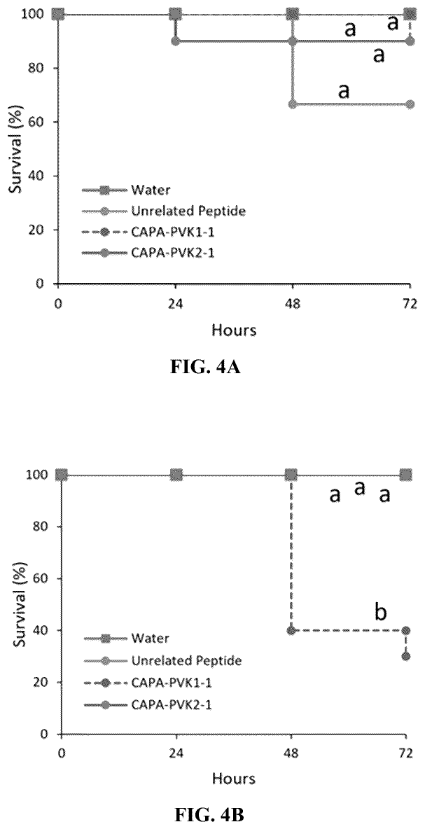

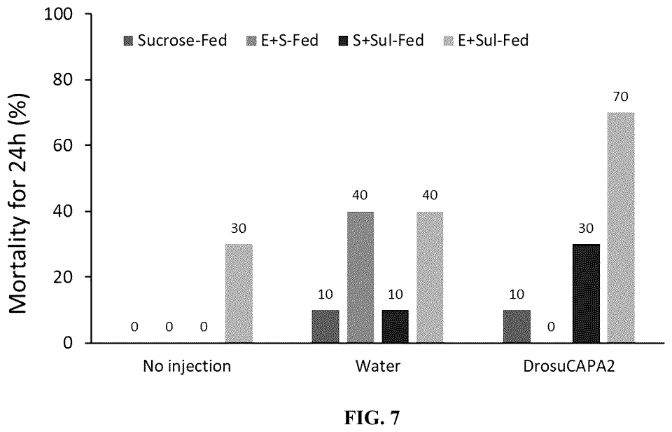

3. Bioactive peptides, when fed to the thrips and SWD flies, have significant lethal effects on thrips (FIGS. 4A and 4B) and SWD (FIG. 6), whereas the water control showed no significant negative effects. The bioactive peptides (CAPA-PVK1-1 (FPFPRV) (SEQ ID NO: 9) and CAPA-PVK2-1 (IPFPRV) (SEQ ID NO: 8) each from Frankliniella occidentalis) showed lethal effect on thrips (FIGS. 4A and 4B), and the bioactive peptide (CAPA-PVK2 (ASGLVAFPRV) (SEQ ID NO: 15) from Drosophila suzukii) increased the lethal effect on the fly after feeding the peptide (FIG. 7)

4. The binding activities of the short peptides to the two GPCRs of the thrips and SWD flies are similar compared to their natural ligands.

5. Knowledge of the structure of the novel peptide agonists facilitates intelligent synthesis of chemical structures with a similar functional group conformation, but with better physical/physiological properties.

Thus, the compositions and methods disclosed herein will have general applicability. The ubiquitous PRXamide GPCRs play a key role in normal development as well as critical adult activities, thus the compositions and methods disclosed herein are extremely versatile as the bioactive peptides negatively affect the normal functioning of both immature and adult insects.

Discovery of bioactive peptides from thrips, fly, moth, and lygus

Screening small/short peptides through four GPCRs biding affinity tests: More than 30 natural ligands and short peptides of insect PRXamide family, have been tested to determine their binding activities to four GPCRs identified from the western flower thrips, spotted-wing drosophila, diamondback moth, and lygus (Tables 1-5, below, as well as examples) expressed in the Sf9 insect cell line.

We identified seven potential short peptides that were measured for their binding affinities based on specific fluorescent intensity of the Sf9 cells in a 96-well plate. A strong fluorescent signal indicates strong binding activity between the peptide ligand and receptors. The EC₅₀ values of all the peptides to the insect receptors were indicated in Tables 1-5, respectively, as determined with in vitro cell line experiments.

Frankliniella occidentalis. Table 1, below, shows the half-maximum effective concentrations (EC₅₀) of the CAPA-PVK and modified peptides tested on the CAPA receptor of Frankliniella occidentalis. Low EC₅₀ values are an indication of strong peptide-receptor binding.

TABLE 1

| | | Amino acid | EC₅₀ |
|---|---|---|---|
| Species (Order) | Peptide | sequence* | value |
| Frankliniella | CAPA-PVK1 | EVQGLFPFPRV | 21 nM |
| occidentalis | | (SEQ ID NO: 1) | |
| (Thysanoptera) | CAPA-PVK2 | QGLIPFPRV | 10 nM |
| | | (SEQ ID NO: 2) | |
| | CAPA-PVK2- | QGLIPFPRV** | 30 nM |
| | acid | (SEQ ID NO: 3) | |
| | CAPA-PVK1- | GLFPFPRV | 3 nM |
| | 3 | (SEQ ID NO: 4) | |
| | CAPA-PVK2- | GLIPFPRV | 9 nM |
| | 3 | (SEQ ID NO: 5) | |
| | CAPA-PVK1- | LFPFPRV | 12 nM |
| | 2 | (SEQ ID NO: 6) | |
| | CAPA-PVK2- | LIPFPRV | 5 nM |
| | 2 | (SEQ ID NO: 7) | |
| | CAPA-PVK2- | IPFPRV | 30 nM |
| | 1 | (SEQ ID NO: 8) | |
| | CAPA-PVK1- | FPFPRV | 24 nM |
| | 1 | (SEQ ID NO: 9) | |
| | PK1 | DLVTQVLQPGQTGVW | >1 μM |
| | (trpPK) | FGPRL | |
| | | (SEQ ID NO: 10) | |
| | CAPA-PK2-1 | VASWMPSSSPRL | >1 μM |
| | | (SEQ ID NO: 11) | |
| | CAPA-PK2-2 | DSASFTPRL | >1 μM |
| | | (SEQ ID NO: 12) | |
| | PK2-1 | SEGNLVNFTPRL | >1 μM |
| | | (SEQ ID NO: 13) | |
| Drosophila | CAPA-PVK1 | GANMGLYAFPRV | 14 nM |
| suzukii | | (SEQ ID NO: 14) | |
| (Diptera) | CAPA-PVK2 | ASGLVAFPRV | 7 nM |
| | | (SEQ ID NO: 15) | |
| Halyomorpha | CAPA-PVK1 | DAGLFPFPRV | 123 nM |
| halys | | (SEQ ID NO: 16) | |
| (Hemiptera) | CAPA-PVK2 | EQLIPFPRV | 87 nM |
| | | (SEQ ID NO: 17) | |
| Helicoverpa | CAPA-PVK1 | DGVLNLYPFPRV | 21 nM |
| zea | | (SEQ ID NO: 18) | |
| (Lepidoptera) | CAPA-PVK2 | QLYAFPRV | 25 nM |
| | | (SEQ ID NO: 19) | |
| Plutella | CAPA-PVK1 | DGVLSLYPFPRV | 40 nM |
| xylostella | | (SEQ ID NO: 20) | |
| (Lepidoptera) | CAPA-PK1 | GSESTDSTSMWFGPR | >1 μM |
| | | L | |
| | | (SEQ ID NO: 21) | |
| Solenopsis | CAPA-PVK1 | SAGLVAYPRI | 532 nM |
| invicta | | (SEQ ID NO: 22) | |
| (Hymenoptera) | CAPA-PVK2 | KSDLFPRL | >1 μM |
| | | (SEQ ID NO: 23) | |
| | CAPA-PVK3 | TFGIIQKPRV | 437 nM |
| | | (SEQ ID NO: 24) | |
| Deroceras | CAPA-PVK1 | VFYTKSDDNDYPRI | >1 μM |
| reticulatum | | (SEQ ID NO: 25) | |
| (Mollusca: | CAPA-PVK2 | GIFTQSAHGSYPRV | >1 μM |
| Stylommatophora) | | (SEQ ID NO: 26) | |

In Table 1:
*All C-terminal ends of the peptides are amidated (NH₂),
**the C-terminal end of the peptide is acidified (COOH),
PVK: periviscerokinin,
ETH: ecdysis triggering hormone,
PK: pyrokinin, PK1 (= DH-like or tryptophan pyrokinin, trpPK), PK2 (= PBAN-like),
DH: diapause hormone.

Drosophila suzukii. Table 2, below, shows the half-maximum effective concentrations (EC₅₀) of the CAPA-PVK and modified peptides tested on the CAPA receptor of Drosophila suzukii. Low EC₅₀ values are an indication of strong peptide-receptor binding.

TABLE 2

| | | Amino acid | EC₅₀ |
|---|---|---|---|
| Species (Order) | Peptide | sequence* | value |
| Frankliniella | CAPA-PVK1 | EVQGLFPFPRV | >1 μM |
| occidentalis | | (SEQ ID NO: 1) | |
| (Thysanoptera) | CAPA-PVK1- | GLFPFPRV | 467 nM |
| | 3 | (SEQ ID NO: 4) | |
| | CAPA-PVK1- | LFPFPRV | 123 nM |
| | 2 | (SEQ ID NO: 6) | |
| | CAPA-PVK1- | FPFPRV | >1 μM |
| | 1 | (SEQ ID NO: 9) | |
| | CAPA-PVK2 | QGLIPFPRV | 49 nM |
| | | (SEQ ID NO: 2) | |
| | CAPA-PVK2- | GLIPFPRV | 129 nM |
| | 3 | (SEQ ID NO: 5) | |
| | CAPA-PVK2- | LIPFPRV | 56 nM |
| | 2 | (SEQ ID NO: 7) | |
| | CAPA-PVK2- | IPFPRV | >1 μM |
| | 1 | (SEQ ID NO: 8) | |
| Drosophila | CAPA-PVK1 | GANMGLYAFPRV | 39 nM |
| suzukii | | (SEQ ID NO: 14) | |
| (Diptera) | CAPA-PVK1- | GLYAFPRV | >1 μM |
| | 3 | (SEQ ID NO: 27) | |
| | CAPA-PVK1- | LYAFPRV | 97 nM |
| | 2 | (SEQ ID NO: 28) | |
| | CAPA-PVK1- | YAFPRV | >1 μM |
| | 1 | (SEQ ID NO: 29) | |
| | CAPA-PVK2 | ASGLVAFPRV | 28 nM |
| | | (SEQ ID NO: 15) | |
| | CAPA-PVK2- | SGLVAFPRV | >1 μM |
| | 4 | (SEQ ID NO: 30) | |
| | CAPA-PVK2- | GLVAFPRV | >1 μM |
| | 3 | (SEQ ID NO: 31) | |
| | CAPA-PVK2- | LVAFPRV | 203 nM |
| | 2 | (SEQ ID NO: 32) | |
| | CAPA-PVK2- | VAFPRV | >1 μM |
| | 1 | (SEQ ID NO: 33) | |
| Halyomorpha | CAPA-PVK1 | DAGLFPFPRV | 546 nM |
| halys | | (SEQ ID NO: 16) | |
| (Hemiptera) | CAPA-PVK2 | EQLIPFPRV | 323 nM |
| | | (SEQ ID NO: 17) | |
| Helicoverpa | CAPA-PVK1 | DGVLNLYPFPRV | 210 nM |
| zea | | (SEQ ID NO: 18) | |
| (Lepidoptera) | CAPA-PVK2 | QLYAFPRV | 163 nM |
| | | (SEQ ID NO: 19) | |
| Plutella | CAPA-PVK1 | DGVLSLYPFPRV | 73 nM |
| xylostella | | (SEQ ID NO: 20) | |
| (Lepidoptera) | | | |
| Solenopsis | CAPA-PVK1 | SAGLVAYPRI | 95 nM |
| invicta | | (SEQ ID NO: 22) | |
| (Hymenoptera) | CAPA-PVK2 | KSDLFPRL | >1 μM |
| | | (SEQ ID NO: 23) | |
| | CAPA-PVK3 | TFGIIQKPRV | 283 nM |
| | | (SEQ ID NO: 24) | |
| Deroceras | CAPA-PVK1 | VFYTKSDDNDYPRI | >1 μM |
| reticulatum | | (SEQ ID NO: 25) | |
| (Mollusca: | | | |
| Stylommatophora) | | | |

In Table 2:
*All C-terminal ends of the peptides are amidated (NH₂),
PVK: periviscerokinin,
ETH: ecdysis triggering hormone,
PK: pyrokinin, PK1 (= DH-like or tryptophan pyrokinin, trpPK), PK2 (= PBAN-like),
DH: diapause hormone.

Plutella xylostella. Table 3, below, shows the half-maximum effective concentrations (EC₅₀) of the CAPA-PVK and modified peptides tested on the CAPA receptor of Plutella xylostella. Low EC₅₀ values are an indication of strong peptide-receptor binding.

TABLE 3

| | | Amino acid | EC₅₀ |
|---|---|---|---|
| Species (Order) | Peptide | sequence* | value |
| Frankliniella | CAPA-PVK1 | EVQGLFPFPRV | 356 nM |
| occidentalis | | (SEQ ID NO: 1) | |
| (Thysanoptera) | CAPA-PVK1- | GLFPFPRV | 66 nM |
| | 3 | (SEQ ID NO: 4) | |
| | CAPA-PVK1- | LFPFPRV | 74 nM |
| | 2 | (SEQ ID NO: 6) | |
| | CAPA-PVK1- | FPFPRV | >1 μM |
| | 1 | (SEQ ID NO: 9) | |
| | CAPA-PVK2 | QGLIPFPRV | 36 nM |
| | | (SEQ ID NO: 2) | |
| | CAPA-PVK2- | GLIPFPRV | 76 nM |
| | 3 | (SEQ ID NO: 5) | |
| | CAPA-PVK2- | LIPFPRV | 110 nM |
| | 2 | (SEQ ID NO: 7) | |
| | CAPA-PVK2- | IPFPRV | >1 μM |
| | 1 | (SEQ ID NO: 8) | |
| Drosophila | CAPA-PVK1 | GANMGLYAFPRV | 145 nM |
| suzukii | | (SEQ ID NO: 14) | |
| (Diptera) | CAPA-PVK1- | GLYAFPRV | 74 nM |
| | 3 | (SEQ ID NO: 27) | |
| | CAPA-PVK1- | LYAFPRV | 306 nM |
| | 2 | (SEQ ID NO: 28) | |
| | CAPA-PVK1- | YAFPRV | >1 μM |
| | 1 | (SEQ ID NO: 29) | |
| | CAPA-PVK2 | ASGLVAFPRV | 109 nM |
| | | (SEQ ID NO: 15) | |
| | CAPA-PVK2- | SGLVAFPRV | 168 nM |
| | 4 | (SEQ ID NO: 30) | |
| | CAPA-PVK2- | GLVAFPRV | 115 nM |
| | 3 | (SEQ ID NO: 31) | |
| | CAPA-PVK2- | LVAFPRV | 149 nM |
| | 2 | (SEQ ID NO: 32) | |
| | CAPA-PVK2- | VAFPRV | >1 μM |
| | 1 | (SEQ ID NO: 33) | |
| Halyomorpha | CAPA-PVK1 | DAGLFPFPRV | 130 nM |
| halys | | (SEQ ID NO: 16) | |
| (Hemiptera) | CAPA-PVK2 | EQLIPFPRV | >1 μM |
| | | (SEQ ID NO: 17) | |
| | CAPA-DH | NGASGNGGLWFGPRL | >1 μM |
| | (PK1) | a | |
| | | (SEQ ID NO: 34) | |
| | ETH1 | NDFFLKAAKSVPRI | >1 μM |
| | | (SEQ ID NO: 35) | |
| Helicoverpa | CAPA-PVK1 | DGVLNLYPFPRV | 231 nM |
| zea | | (SEQ ID NO: 18) | |
| (Lepidoptera) | CAPA-PVK2 | QLYAFPRV | 194 nM |
| | | (SEQ ID NO: 19) | |
| | PBAN | LSD...YFSPRLa | >1 μM |
| | | (SEQ ID NO: 36) | |
| Plutella | CAPA-PVK1 | DGVLSLYPFPRV | 635 nM |
| xylostella | | (SEQ ID NO: 20) | |
| (Lepidoptera) | CAPA-PK1 | GSESTDSTSMWFGPR | >1 μM |
| | | L | |
| | | (SEQ ID NO: 21) | |
| Solenopsis | CAPA-PVK1 | SAGLVAYPRI | >1 μM |
| invicta | | (SEQ ID NO: 22) | |
| (Hymenoptera) | CAPA-PVK2 | KSDLFPRL | >1 μM |
| | | (SEQ ID NO: 23) | |
| | CAPA-PVK3 | TFGIIQKPRV | >1 μM |
| | | (SEQ ID NO: 24) | |
| Deroceras | CAPA-PVK1 | VFYTKSDDNDYPRI | >1 μM |
| reticulatum | | (SEQ ID NO: 25) | |
| (Mollusca: | CAPA-PVK2 | GIFTQSAHGSYPRV | >1 μM |
| Stylommatophora) | | (SEQ ID NO: 26) | |

In Table 3:
*All C-terminal ends of the peptides are amidated (NH₂),
PVK: periviscerokinin,
ETH: ecdysis triggering hormone,
PK: pyrokinin, PK1 (= DH-like or tryptophan pyrokinin, trpPK), PK2 (= PBAN-like),
DH: diapause hormone.

Lygus hesperus. Table 4, below, shows the half-maximum effective concentrations (EC₅₀) of the CAPA-PVK and modified peptides tested on the CAPA receptor of Lygus hesperus. Low EC₅₀ values are an indication of strong peptide-receptor binding.

TABLE 4

| | | Amino acid | EC₅₀ |
|---|---|---|---|
| Species (Order) | Peptide | sequence* | value |
| Frankliniella | CAPA- | EVQGLFPFPRV | >1 μM |
| occidentalis | PVK1 | (SEQ ID NO: 1) | |
| (Thysanoptera) | CAPA- | GLFPFPRV | 20 nM |
| | PVK1-3 | (SEQ ID NO: 4) | |
| | CAPA- | LFPFPRV | 21 nM |
| | PVK1-2 | (SEQ ID NO: 6) | |
| | CAPA- | FPFPRV | 29 nM |
| | PVK1-1 | (SEQ ID NO: 9) | |
| | CAPA- | QGLIPFPRV | >1 μM |
| | PVK2 | (SEQ ID NO: 2) | |
| | CAPA- | GLIPFPRV | 17 nM |
| | PVK2-3 | (SEQ ID NO: 5) | |
| | CAPA- | LIPFPRV | 7 nM |
| | PVK2-2 | (SEQ ID NO: 7) | |
| | CAPA- | IPFPRV | 52 nM |
| | PVK2-1 | (SEQ ID NO: 8) | |
| Drosophila | CAPA- | GANMGLYAFPRV | >1 μM |
| suzukii | PVK1 | (SEQ ID NO: 14) | |
| (Diptera) | CAPA- | GLYAFPRV | 104 nM |
| | PVK1-3 | (SEQ ID NO: 27) | |
| | CAPA- | LYAFPRV | 17 nM |
| | PVK1-2 | (SEQ ID NO: 28) | |
| | CAPA- | YAFPRV | 16 nM |
| | PVK1-1 | (SEQ ID NO: 29) | |
| | CAPA- | ASGLVAFPRV | >1 μM |
| | PVK2 | (SEQ ID NO: 15) | |
| | CAPA- | SGLVAFPRV | 49 nM |
| | PVK2-4 | (SEQ ID NO: 30) | |
| | CAPA- | GLVAFPRV | 16 nM |
| | PVK2-3 | (SEQ ID NO: 31) | |
| | CAPA- | LVAFPRV | 27 nM |
| | PVK2-2 | (SEQ ID NO: 32) | |
| | CAPA- | VAFPRV | 19 nM |
| | PVK2-1 | (SEQ ID NO: 33) | |
| Halyomorpha | CAPA- | DAGLFPFPRV | >1 μM |
| halys | PVK1 | (SEQ ID NO: 16) | |
| (Hemiptera) | CAPA- | EQLIPFPRV | 29 nM |
| | PVK2 | (SEQ ID NO: 17) | |
| | CAPA-DH | NGASGNGGLWFGPRL | >1 μM |
| | (PK1) | a | |
| | | (SEQ ID NO: 34) | |
| | ETH1 | NDFFLKAAKSVPRI | >1 μM |
| | | (SEQ ID NO: 35) | |
| Lygus Hesperus | CAPA- | DTSGLIPFPRV | 22 nM |
| (Hemiptera) | PVK | (SEQ ID NO: 37) | |
| Helicoverpa | CAPA- | DGVLNLYPFPRV | >1 μM |
| zea | PVK1 | (SEQ ID NO: 18) | |
| (Lepidoptera) | CAPA- | QLYAFPRV | 66 nM |
| | PVK2 | (SEQ ID NO: 19) | |
| | PBAN | LSD...YFSPRLa | >1 μM |
| | | (SEQ ID NO: 36) | |
| Plutella | CAPA- | DGVLSLYPFPRV | >1 μM |
| xylostella | PVK1 | (SEQ ID NO: 20) | |
| (Lepidoptera) | | | |
| Solenopsis | CAPA- | SAGLVAYPRI | 80 nM |
| invicta | PVK1 | (SEQ ID NO: 22) | |
| (Hymenoptera) | CAPA- | KSDLFPRL | >1 μM |
| | PVK2 | (SEQ ID NO: 23) | |
| | CAPA- | TFGIIQKPRV | >1 μM |
| | PVK3 | (SEQ ID NO: 24) | |
| Deroceras | CAPA- | VFYTKSDDNDYPRI | >1 μM |
| reticulatum | PVK1 | (SEQ ID NO: 25) | |
| (Mollusca: | CAPA- | GIFTQSAHGSYPRV | >1 μM |
| Stylommatophora) | PVK2 | (SEQ ID NO: 26) | |

In Table 4:
*All C-terminal ends of the peptides are amidated (NH₂),
PVK: periviscerokinin,
ETH: ecdysis triggering hormone,
PK: pyrokinin, PK1 (= DH-like or tryptophan pyrokinin, trpPK), PK2 (= PBAN-like),
DH: diapause hormone.

Table 5, below, is a list of structurally modified peptides for testing on various insect CAPA receptors and/or the thrips feeding assay.

TABLE 5

| Peptide | Amino acid sequence* |
|---|---|
| CAPA-PFPRV | PFPRV |
| | (SEQ ID NO: 38) |
| CAPA-AFPRV | AFPR |
| | (SEQ ID NO: 39) |
| CAPA-FPRV | FPRV |
| | (SEQ ID NO: 40) |
| CAPA-PRV | PRV |
| | (SEQ ID NO: 41) |
| CAPA1-1C | CFPFPRVC |
| | (SEQ ID NO: 42) |
| CAPA1-2C | CLFPFPRVC |
| | (SEQ ID NO: 43) |
| CAPA1-3C | CGLFPFPRVC |
| | (SEQ ID NO: 44) |
| CAPA2-1C | CIPFPRVC |
| | (SEQ ID NO: 45) |
| CAPA2-2C | CLIPFPRVC |
| | (SEQ ID NO: 46) |
| CAPA2-3C | CGLIPFPRVC |
| | (SEQ ID NO: 47) |
| CAPA-PVK2-acid | QGLIPFPRV |
| | (SEQ ID NO: 3) |

In Table 5:
*All C-terminal ends of the peptides are amidated (NH₂). CAPA-PVK2-acid is acidified (COOH) in the C-terminus.

### EXAMPLES

### Example 1

### In Vitro Assay of Thrips Peptides

As shown in FIGS. 1A and 1B, thrips peptides (CAPA-PVK1 (EVQGLFPFPRV) (SEQ ID NO:1) (labeled in FIG. 1A as FraocCAPA1) and CAPA-PVK2 (QGLIPFPRV) (SEQ ID NO: 2) (labeled in FIG. 1B as FraocCAPA2)) from Frankliniella occidentalis, as well as their respective modified peptides (CAPA-PVK1-1 (FPFPRV) (SEQ ID NO: 9) (labeled in FIG. 1A as FraocCAPA1-1), CAPA-PVK1-2 (LFPFPRV) (SEQ ID NO: 6)) (labeled in FIG. 1A as FraocCAPA1-2), and CAPA-PVK1-3 (GLFPFPRV) (SEQ ID NO: 4)) (labeled in FIG. 1A as FraocCAPA1-3); and CAPA-PVK2-1 (IPFPRV) (SEQ ID NO: 8)) (labeled in FIG. 1B as FraocCAPA2-1), CAPA-PVK2-2 (LIPFPRV) (SEQ ID NO: 7)) (labeled in FIG. 1B as FraocCAPA2-2), and CAPA-PVK2-3 (GLIPFPRV) (SEQ ID NO: 5)) (labeled in FIG. 1B as FraocCAPA2-3)) will be agonists of the natural peptide ligands for thrips receptors and interfere with the normal functioning of the peptides and their receptors. FIG. 1A shows dosage-response of CAPA-PVK1 (EVQGLFPFPRV) (SEQ ID NO: 1) (labeled in FIG. 1A as FraocCAPA1) and its modified bioactive peptides (CAPA-PVK1-1 (FPFPRV) (SEQ ID NO: 9) (labeled in FIG. 1A as FraocCAPA1-1), CAPA-PVK1-2 (LFPFPRV) (SEQ ID NO: 6) (labeled in FIG. 1A as FraocCAPA1-2), and CAPA-PVK1-3 (GLFPFPRV) (SEQ ID NO: 4) (labeled in FIG. 1A as FraocCAPA1-3)) on the CAPA GPCR identified from western flower thrips, Frankliniella occidentalis. FIG. 1B shows dosage-response of CAPA-PVK2 (QGLIPFPRV) (SEQ ID NO: 2) (labeled in FIG. 1B as FraocCAPA2) and its modified bioactive peptides (CAPA-PVK2-1 (IPFPRV) (SEQ ID NO: 8) (labeled in FIG. 1B as FraocCAPA2-1), CAPA-PVK2-2 (LIPFPRV) (SEQ ID NO: 7) (labeled in FIG. 1B as FraocCAPA2-1), and CAPA-PVK2-3 (GLIPFPRV) (SEQ ID NO: 5) (labeled in FIG. 1B as FraocCAPA2-3)) on the CAPA GPCR identified from western flower thrips, Frankliniella occidentalis.

### Example 2

Injection of Peptides into Thrips

Twelve female adults were injected into the thoracic region with a 10 nl (5 nl/see; 20 pmol) of treatment solutions: CAPA-PVK1-1 (FPFPR-NH2) (SEQ ID NO: 9), CAPA-PVK1-2 (LFPFPR-NH2) (SEQ ID NO: 6), CAPA-PVK2-1 (IPFPRV-NH2) (SEQ ID NO: 8), an unrelated peptide from Meloidogyne incognita (PGVLRF-NH2), or water as a control, using a NANOLITER2020 Injector. After 30 min, mortality was recorded and remaining thrips were provided a ⅛^{th} piece of cotyledon. Mortality was checked again after 24 hours. To analyze mortality by treatment, individuals were marked yes/no, and a contingency chi-square analysis was conducted in JMP Pro 15.1 at 1 hour and 24 hours after injection.

As shown in FIG. 2, when peptides were injected into thrips, modalities of thrips showed 55% from CAPA-PVK2 (QGLIPFPRV) (SEQ ID NO: 2) (labeled in FIG. 2 as FraocCAPA2) and 93% from CAPA-PK2-1 (VASWMPSSSPRL) (SEQ ID NO: 11) (labeled in FIG. 2 as FraocPK2-1) (P<0.05) for 24 h, however, the control was 10% mortality. FIG. 2 shows mortality of female thrips within 24 h after injection of bioactive peptides of western flower thrips, Frankliniella occidentalis. FraocCAPA2 (1 nmol) or FraocPK2-1 (0.5 nmol) were dissolved in 5 nL water. Control: 5 nL water. Three replicates per each treatment.

### Example 3

### Feeding Peptides to Thrips

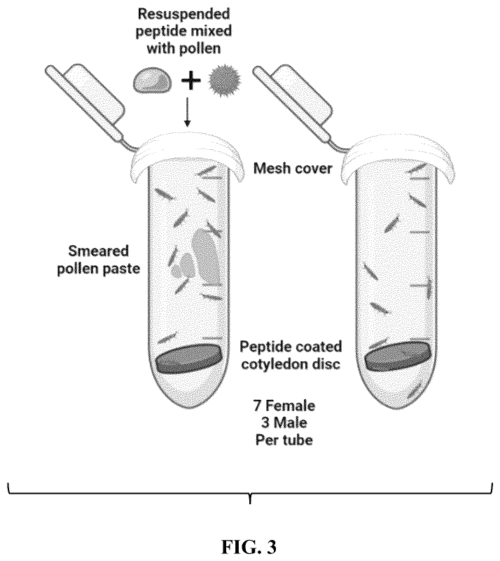

FIG. 3 depicts feeding assay with and without pollen as a phagostimulant in thrips, according to one or more embodiments.

Thrips were starved for 1 hour before feeding. Seven female and three male adults were transferred into 2 ml centrifuge tubes (FIG. 3, right side). Using a metal straw, 7 mm discs were punctured from the center of soybean cotyledons. The resuspended peptide (10 μl; 10 nmol) was pipetted onto aluminum foil and cotyledon discs were rolled in the droplet using forceps until generously coated with CAPA-PVK1-1 (FPFPRV) (SEQ ID NO: 9), CAPA-PVK2-1 (IPFPRV) (SEQ ID NO: 8), an unrelated peptide from M. incognita or water as a control. Discs were allowed to dry for 20 minutes, then introduced to thrips.

Since thrips have piercing-sucking mouthparts and would mostly be consuming plant tissue under the epidermis of the cotyledon, an alternate delivery method may be necessary for thrips to consume a lethal amount of peptide. A second preliminary trial was done using a cotyledon disc as described above with the addition of 10 nmol peptide-treated pollen (=20 nmol total in tubes) (FIG. 3, left side). Pollen is known to stimulate feeding and heighten fitness of thrips (Kirk 1984). For this trial, bee pollen grains (Mickelberry Gardens, Portland, OR) were weighed (9-10 mg), crushed using a spatula, and mixed with 10 μl of CAPA-PVK1-1 (FPFPRV) (SEQ ID NO: 9), CAPA-PVK2-1 (IPFPRV) (SEQ ID NO: 8), an unrelated peptide from M. incognita or water as a control. The pollen paste was smeared onto one wall of a 2 ml centrifuge tube containing a treated cotyledon disc and allowed to dry for 10 minutes. Then, 7 female and 3 male thrips were introduced to the tube.

Both preliminary trials were kept at 25±1° C., 12 L: 12D photoperiod, 60% relative humidity and analyzed the same way. Mortality was monitored daily for 72 h days. Survival rates were compared between treatments by log-rank analysis in Proc Lifetest in SAS 9.4 (SAS Institute 2016). Multiple comparisons were done using an adjusted Šidák P-value. The remaining survivors at 72 h were censored data.

FIGS. 4A and 4B depicts survival (%) of Frankliniella occidentallis over 72 hours when fed various peptide solutions from two different delivery methods, according to one or more embodiments. FIG. 4A shows survival (%) of Frankliniella occidentallis over 72 hours when fed various peptide solutions without pollen as a phagostimulant. FIG. 4B shows survival (%) of Frankliniella occidentallis over 72 hours when fed various peptide solutions with pollen as a phagostimulant.

As shown in FIG. 4A, when thrips were given a peptide-treated cotyledon only, survival of injected thrips did not differ among treatments (χ2=3.229, df=3, p=0.357) with some mortality (10-25%) when fed the unrelated peptide or CAPA-PVK2-1 (IPFPRV) (SEQ ID NO: 8). As shown in FIG. 4B, when provided the additional peptide-treated pollen paste, there was a significant difference in survival by treatment (χ2=25.142, df=3, p<0.0001), with survival rates at ˜40% within 48 h and at 30% within 72 h from thrips fed CAPA-PVK1-1 (FPFPRV) (SEQ ID NO: 9), respectively. Thrips fed CAPA-PVK2-1 (IPFPRV) (SEQ ID NO: 8), the unrelated nematode peptide and water fared similarly, with no mortality in the 72 h.

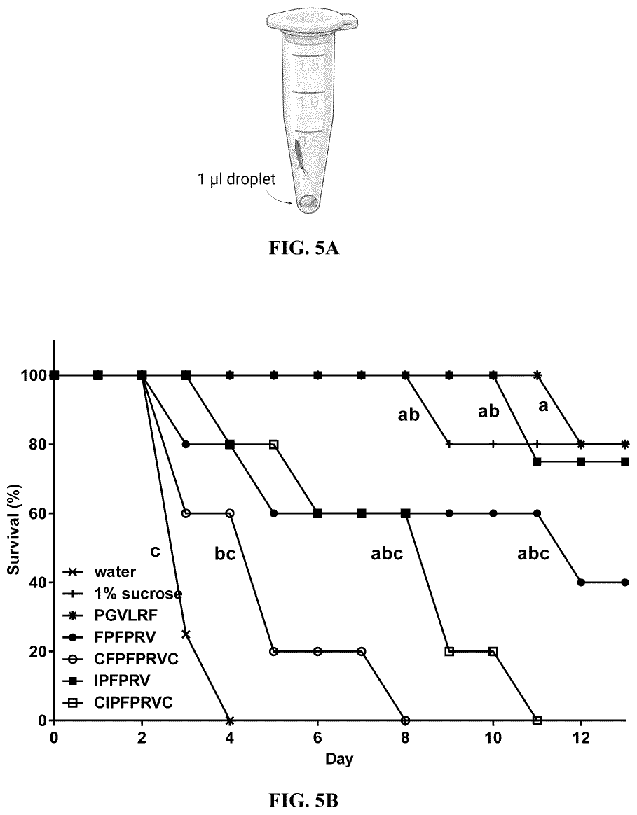

FIGS. 5A and 5B respectively depict a feeding assay with bioactive peptides on thrips (FIG. 5A) and percentage of thrips survival (FIG. 5B), according to one or more embodiments. Thrips fed on 1% sucrose solution containing 10 nmol of bioactive peptide and mortality was checked daily for two weeks. The bioactive peptides contained in the droplet of 1% sucrose solution included CAPA-PVK1-1 (FPFPRV) (SEQ ID NO: 9), CAPA1-1C (CFPFPRVC) (SEQ ID NO: 42), CAPA-PVK2-1 (IPFPRV) (SEQ ID NO: 8), CAPA2-1C (CIPFPRVC) (SEQ ID NO: 45), or an unrelated peptide from Meloidogyne incognita (PGVLRF). The droplet of solution typically lasts about 5 days. The droplet of solution normally lasted 5 days and was replaced with a new droplet of solution, but was checked daily to decide whether to replace the droplet before or after 5 days. Water only without sucrose was used as negative control. Thrips were starved 1 h prior to feeding. Five replicates. Survival rates were compared between treatments using log-rank analysis in SAS 9.4.

### Example 4

### In Vitro Assay of SWD Peptides

FIG. 6 depicts dosage-response of CAPA peptides on the CAPA GPCR identified from spotted-wing drosophila (SWD), Drosophila suzukii, according to one or more embodiments. As shown in FIG. 6, SWD peptides (CAPA-PVK1 (GANMGLYAFPRV) (SEQ ID NO: 14) (labeled in FIG. 6 as DrosuCAPA1) and CAPA-PVK2 (ASGLVAFPRV) (SEQ ID NO: 15) (labeled in FIG. 6 as DrosuCAPA1)) from Drosophila suzukii, as well as thrip peptide (CAPA-PVK2 (QGLIPFPRV) (SEQ ID NO: 2) (labeled in FIG. 6 as FraocCAPA2)) from Frankliniella occidentalis will be agonists of the natural peptide ligands for SWD receptors and interfere with the normal functioning of the peptides and their receptors.

### Example 5

### Feeding Peptides to SWD

FIG. 7 depicts mortality of spotted-wing drosophila (Drosophila suzukii) adults within 24 h after the feeding of the non-nutritive sugars and injecting the bioactive peptide, according to one or more embodiments. The flies were injected with a 100 pmol of the bioactive peptide CAPA-PVK2 (ASGLVAFPRV) (SEQ ID NO: 15) (labeled in FIG. 7 as DrosuCAPA2) after 1 h feeding of the diets and fed continuously for 24 h. S: 0.5M sucrose; E+S: 0.5M erythritol+0.5M sucrose; S+Sul: 0.5M sucrose+0.5M sucralose; E+Sul: 0.5M erythritol+0.5M sucralose.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. An insecticide composition for controlling an insect, the composition comprising an effective amount of peptide having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, SEQ ID NO: 42, SEQ ID NO: 45, or a combination thereof.

2. The composition of claim 1, further comprising an insecticide carrier.

3. The composition of claim 2, wherein the carrier is saline.

4. The composition of claim 1, further comprising one or more suitable propellants, carriers, diluents, adjuvants, preservative, dispersants, solvents, or emulsifying agents.

5. The composition of claim 1, wherein the peptide has SEQ ID NO: 4, SEQ ID NO:
5, SEQ ID NO: 6, SEQ ID NO: 7, SEQ ID NO: 8, SEQ ID NO: 9, SEQ ID NO: 28, or a combination thereof.

6. The composition of claim 1, wherein the peptide has SEQ ID NO:4.

7. The composition of claim 1, wherein the peptide has SEQ ID NO:5.

8. The composition of claim 1, wherein the peptide has SEQ ID NO:6.

9. The composition of claim 1, wherein the peptide has SEQ ID NO:7.

10. The composition of claim 1, wherein the peptide has SEQ ID NO:8.

11. The composition of claim 1, wherein the peptide has SEQ ID NO:9.

12. The composition of claim 1, wherein the peptide has SEQ ID NO:28.

13. A spray composition for controlling an insect, comprising:
a) an effective amount of the composition of claim 1,
b) a propellant.

14. A bait composition for controlling an insect, comprising:
a) an effective amount of the composition of claim 1,
b) one or more food materials;
c) optionally a phagostimulant.

15. A method for controlling an insect comprising contacting an insect or its environment with a biologically effective amount of a composition of claim 1, wherein the mortality of the insect increases.

16. The method of claim 15, wherein the insect is a Frankliniella ssp.

17. The method of claim 16, wherein the peptide has SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 42, SEQ ID NO: 45, or a combination thereof.

18. The method of claim 17, wherein the insect is Frankliniella occidentalis.

19. The method of claim 15, wherein the insect is a Drosophila ssp.

20. The method of claim 19, wherein the peptide has SEQ ID NO: 2, SEQ ID NO: 4, SEQ ID NO: 5, SEQ ID NO: 6, SEQ ID NO: 7, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 24, SEQ ID NO: 28, SEQ ID NO: 32, or a combination thereof.

21. The method of claim 20, wherein the insect is Drosophila suzukii.

22. The method of claim 15, wherein the insect is a Plutella ssp.

23. The method of claim 22, wherein the wherein the peptide has SEQ ID NO: 1, SEQ ID NO: 2, SEQ ID NO: 4, SEQ ID NO: 5, SEQ ID NO: 6, SEQ ID NO: 7, SEQ ID NO: 14, SEQ ID NO: 15, SEQ ID NO: 16, SEQ ID NO: 18, SEQ ID NO: 19, SEQ ID NO: 20, SEQ ID NO: 27, SEQ ID NO: 28, SEQ ID NO: 30, SEQ ID NO: 31, SEQ ID NO: 32, or a combination thereof.

24. The method of claim 23, wherein the insect is Plutellaxylostella.

25. The method of claim 15, wherein the insect is a Lygus ssp.

26. The method of claim 25, wherein the wherein the peptide has SEQ ID NO: 4, SEQ ID NO: 5, SEQ ID NO: 6, SEQ ID NO: 7, SEQ ID NO: 8, SEQ ID NO: 9, SEQ ID NO: 17, SEQ ID NO: 19, SEQ ID NO: 22, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 37, or a combination thereof.

27. The method of claim 26, wherein the insect is Lygushesperus.

28. An insecticide composition for controlling an insect, the composition comprising an effective amount of peptide having SEQ ID NO: 38, SEQ ID NO: 39, SEQ ID NO: 40, SEQ ID NO: 41, SEQ ID NO: 43, SEQ ID NO: 44, SEQ ID NO: 46, SEQ ID NO: 47, or a combination thereof.
